SEQUENCE LISTING (103 sequences)

SEQ ID NO: 1 - protein, 13 aa, Homo sapiens
CASSMETGNTIYF

SEQ ID NO: 2 - protein, 14 aa, Homo sapiens
CASSLEERNYGYTF

SEQ ID NO: 3 - protein, 14 aa, Homo sapiens
CASSSKGGYQPQHF

SEQ ID NO: 4 - protein, 13 aa, Homo sapiens
CASRTGGNYGYTF

SEQ ID NO: 5 - protein, 13 aa, Homo sapiens
CAISESVYYEQYF

SEQ ID NO: 6 - protein, 16 aa, Homo sapiens
CASSYSAQFSGNTIYF

SEQ ID NO: 7 - protein, 14 aa, Homo sapiens
CASSRGDSNYGYTF

SEQ ID NO: 8 - protein, 13 aa, Homo sapiens
CAWTPKGRETQYF

SEQ ID NO: 9 - protein, 13 aa, Homo sapiens
CASKRVPNTEAFF

SEQ ID NO: 10 - protein, 12 aa, Homo sapiens
CSGTGGGSGYTF

SEQ ID NO: 11 - protein, 12 aa, Homo sapiens
CASSLGGTEQYF

SEQ ID NO: 12 - protein, 15 aa, Homo sapiens
CASSLDRPSNQPQHF

SEQ ID NO: 13 - protein, 14 aa, Homo sapiens
CASSLEGGTDTQYF

SEQ ID NO: 14 - protein, 14 aa, Homo sapiens
CASSLNWNNYGYTF

SEQ ID NO: 15 - protein, 15 aa, Homo sapiens
CASSPRRADTYEQYF

SEQ ID NO: 16 - protein, 20 aa, Homo sapiens
CASSLLHMFTPNSGANVLTF

SEQ ID NO: 17 - protein, 15 aa, Homo sapiens
CASSLDGAWENGYTF

SEQ ID NO: 18 - protein, 13 aa, Homo sapiens
CASISDTYNEQFF

SEQ ID NO: 19 - DNA, 60 bp, Homo sapiens
cagacatctgtgtacttctgtgccagcagtatggagactggaaacaccatatattttgga

SEQ ID NO: 20 - DNA, 66 bp, Homo sapiens
ggggactcggccctctatctctgtgccagcagcttggaagaaaggaactatggctacaccttcggt

SEQ ID NO: 21 - DNA, 63 bp, Homo sapiens
cagacatctgtgtacttctgtgccagcagctccaagggaggttatcagccccagcattttggt

SEQ ID NO: 22 - DNA, 63 bp, Homo sapiens
gacgactcggccctgtatctctgtgccagcaggacaggggggaactatggctacaccttcggt

SEQ ID NO: 23 - DNA, 66 bp, Homo sapiens
agctcccagacatctgtgtacttctgtgccatcagtgagtccgtctactacgagcagtacttcggg

SEQ ID NO: 24 - DNA, 69 bp, Homo sapiens
cagacatctgtgtacttctgtgccagcagttactctgcgcagttctctggaaacaccatatattttgga

SEQ ID NO: 25 - DNA, 66 bp, Homo sapiens
aaccagacctctctgtacttctgtgccagcagtcgaggggacagtaactatggctacaccttcggt

SEQ ID NO: 26 - DNA, 66 bp, Homo sapiens
ctcagtgactctggcttctatctctgtgcctggactccaaaaggacgagagacccagtacttcggg

SEQ ID NO: 27 - DNA, 63 bp, Homo sapiens
aaccagacctctctgtacttctgtgccagcaaacgagttccgaacactgaagctttctttgga

SEQ ID NO: 28 - DNA, 63 bp, Homo sapiens
cctgaagacagcagcttctacatctgcagtgggacagggggcgggagtggctacaccttcggt

SEQ ID NO: 29 - DNA, 60 bp, Homo sapiens
ggggactcggccctctatctctgtgccagcagcttgggtgggaccgagcagtacttcggg

SEQ ID NO: 30 - DNA, 69 bp, Homo sapiens
aaccagacctctctgtacttctgtgccagcagtttagacagaccctccaatcagccccagcattttggt

SEQ ID NO: 31 - DNA, 66 bp, Homo sapiens
ggggactcggccatgtatctctgtgccagcagcttggaggggggaacagatacgcagtattttggc

SEQ ID NO: 32 - DNA, 66 bp, Homo sapiens
aaccagacatctatgtacctctgtgccagcagtttgaattggaataactatggctacaccttcggt

SEQ ID NO: 33 - DNA, 69 bp, Homo sapiens
aacccgacagctttctatctctgtgccagtagtccccgaagggccgacacctacgagcagtacttcggg

SEQ ID NO: 34 - DNA, 84 bp, Homo sapiens
aacccgacagctttctatctctgtgccagtagcctactacacatgtttacccccaactctggggccaacgtcctgactttcggg

SEQ ID NO: 35 - DNA, 69 bp, Homo sapiens
ggggactcggccctttatctttgcgccagcagcctggacggtgcgtgggaaaatggctacaccttcggt

SEQ ID NO: 36 - DNA, 63 bp, Homo sapiens
aacccgacagctttctatctctgtgccagtatttcggacacatacaatgagcagttcttcggg

SEQ ID NO: 37 - protein, 13 aa, Homo sapiens
CASSFEGNYGYTF

SEQ ID NO: 38 - protein, 14 aa, Homo sapiens
CASSFSPSGNTIYF

SEQ ID NO: 39 - protein, 14 aa, Homo sapiens
CASRSDSMNTEAFF

SEQ ID NO: 40 - protein, 13 aa, Homo sapiens
CASSLAFSNEQFF

SEQ ID NO: 41 - protein, 17 aa, Homo sapiens
CASSQEARTSGGETQYF

SEQ ID NO: 42 - protein, 15 aa, Homo sapiens
CASSLDEGISYGYTF

SEQ ID NO: 43 - protein, 12 aa, Homo sapiens
CATGSYNSPLHF

SEQ ID NO: 44 - protein, 13 aa, Homo sapiens
CASSPRDRYEAFF

SEQ ID NO: 45 - protein, 16 aa, Homo sapiens
CASSPLPTGADTEAFF

SEQ ID NO: 46 - protein, 13 aa, Homo sapiens
CASSLEKGQPQHF

SEQ ID NO: 47 - protein, 14 aa, Homo sapiens
CASSRRGMNTEAFF

SEQ ID NO: 48 - protein, 14 aa, Homo sapiens
CASRDSGGNSPLHF

SEQ ID NO: 49 - protein, 12 aa, Homo sapiens
CSVLTPNYGYTF

SEQ ID NO: 50 - protein, 16 aa, Homo sapiens
CASRAGHTVTQIGYTF

SEQ ID NO: 51 - protein, 16 aa, Homo sapiens
CASSGPGVSGANVLTF

SEQ ID NO: 52 - protein, 16 aa, Homo sapiens
CASSYGSGQGFVGYTF

SEQ ID NO: 53 - protein, 14 aa, Homo sapiens
CASSFRTRSYEQYF

SEQ ID NO: 54 - protein, 15 aa, Homo sapiens
CASSAATVGDTEAFF

SEQ ID NO: 55 - protein, 12 aa, Homo sapiens
CASSIHTSEAFF

SEQ ID NO: 56 - protein, 15 aa, Homo sapiens
CASSLGASGGDEQFF

SEQ ID NO: 57 - protein, 12 aa, Homo sapiens
CAARGNTDTQYF

SEQ ID NO: 58 - protein, 16 aa, Homo sapiens
CASRRQGVQLGTEAFF

SEQ ID NO: 59 - protein, 15 aa, Homo sapiens
CASSYSRDIGTEAFF

SEQ ID NO: 60 - protein, 17 aa, Homo sapiens
CASSSPAWGGDQNIQYF

SEQ ID NO: 61 - protein, 13 aa, Homo sapiens
CASSLNNNYGYTF

SEQ ID NO: 62 - protein, 16 aa, Homo sapiens
CASSNGPNVPTDTQYF

SEQ ID NO: 63 - protein, 15 aa, Homo sapiens
CASSLAAGGYNEQFF

SEQ ID NO: 64 - protein, 14 aa, Homo sapiens
CASRWTGGRDTQYF

SEQ ID NO: 65 - protein, 15 aa, Homo sapiens
CASSLVQTGSRGYTF

SEQ ID NO: 66 - protein, 13 aa, Homo sapiens
CASSLPGKTEAFF

SEQ ID NO: 67 - protein, 14 aa, Homo sapiens
CASSVGGRGGTQYF

SEQ ID NO: 68 - protein, 15 aa, Homo sapiens
CASRLSGGAGGEQFF

SEQ ID NO: 69 - protein, 15 aa, Homo sapiens
CASREETGPNTEAFF

SEQ ID NO: 70 - protein, 14 aa, Homo sapiens
CASSDSLTVPGYTF

SEQ ID NO: 71 - protein, 14 aa, Homo sapiens
CASSYRGMNTEAFF

SEQ ID NO: 72 - protein, 13 aa, Homo sapiens
CAWSVWTGDGYTF

SEQ ID NO: 73 - protein, 14 aa, Homo sapiens
CASSVWRLEETQYF

SEQ ID NO: 74 - protein, 15 aa, Homo sapiens
CASSYRDRGRDGYTF

SEQ ID NO: 75 - protein, 14 aa, Homo sapiens
CASSILGRNTEAFF

SEQ ID NO: 76 - protein, 11 aa, Homo sapiens
CASSTYTEAFF

SEQ ID NO: 77 - protein, 14 aa, Homo sapiens
CASSFGGANYGYTF

SEQ ID NO: 78 - protein, 12 aa, Homo sapiens
CASSSRARGYTF

SEQ ID NO: 79 - protein, 15 aa, Homo sapiens
CATSDRVLSNQPQHF

SEQ ID NO: 80 - protein, 13 aa, Homo sapiens
CASSFLPNYGYTF

SEQ ID NO: 81 - protein, 13 aa, Homo sapiens
CASKRASTDEQYF

SEQ ID NO: 82 - protein, 13 aa, Homo sapiens
CASSELGNTEAFF

SEQ ID NO: 83 - protein, 16 aa, Homo sapiens
CASSSTPDRRGYEQYF

SEQ ID NO: 84 - protein, 15 aa, Homo sapiens
CATNPQGNPTYEQYF

SEQ ID NO: 85 - protein, 15 aa, Homo sapiens
CASTSRNSGTYEQYF

SEQ ID NO: 86 - protein, 16 aa, Homo sapiens
CASRRTGGSGANVLTF

SEQ ID NO: 87 - protein, 11 aa, Homo sapiens
CASNVMGRYTF

SEQ ID NO: 88 - protein, 15 aa, Homo sapiens
CASSRLVASTDTQYF

SEQ ID NO: 89 - protein, 14 aa, Homo sapiens
CASRVTSHHYEQYF

SEQ ID NO: 90 - protein, 16 aa, Homo sapiens
CSARDTLRQGAREQYF

SEQ ID NO: 91 - protein, 11 aa, Homo sapiens
CASSLWGTQYF

SEQ ID NO: 92 - protein, 16 aa, Homo sapiens
CASSSSQQGPDYGYTF

SEQ ID NO: 93 - protein, 13 aa, Homo sapiens
CSALGLPTDTQYF

SEQ ID NO: 94 - protein, 12 aa, Homo sapiens
CASSVRARGYTF

SEQ ID NO: 95 - DNA, 39 bp, Homo sapiens
tgtgccagcagcttaaacaataactatggctacaccttc

SEQ ID NO: 96 - DNA, 51 bp, Homo sapiens
tgtgccagcagttccccagcttggggcggggaccaaaacattcagtacttc

SEQ ID NO: 97 - DNA, 39 bp, Homo sapiens
tgtgccagcagtatggagactggaaacaccatatatttt

SEQ ID NO: 98 - DNA, 30 bp, Homo sapiens
tgtgcaggaggttccaatgacatgcgcttt

SEQ ID NO: 99 - DNA, 45 bp, Homo sapiens
tgcatcgtcacgaggattggaggtagcaactataaactgacattt

SEQ ID NO: 100 - DNA, 45 bp, Homo sapiens
tgtgccgtgaattcagttgggggaagccaaggaaatctcatcttt

SEQ ID NO: 101 - DNA, 20 bp, Artificial Sequence
  Primer
cttctccttctccagcacca

SEQ ID NO: 102 - DNA, 20 bp, Artificial Sequence
  Primer
gacaccatttgccagcagtg

SEQ ID NO: 103 - DNA, 20 bp, Artificial Sequence
  Primer
ttgagggagaagaccccagt

(19) United States
(12) Patent Application Publication
(10) Pub. No.: US 2022/0143083 A1
(43) Pub. Date: May 12, 2022

(54) REVERSE IMMUNOSUPPRESSION

(71) Applicant: THERYCELL GMBH, Berlin (DE)

(72) Inventors: Rudolf HAMMER, Zornheim (DE); Steffen HENNIG, Berlin (DE)

(73) Assignee: THERYCELL GMBH, Berlin (DE)

(21) Appl. No.: 17/429,996

(22) PCT Filed: Feb. 12, 2020
(86) PCT No.: PCT/EP2020/053648

§ 371 Date: Aug. 11, 2021

(30) Foreign Application Priority Data
Feb. 12, 2019 (EP) 19156745.2

(51) Int. Cl. A61K 35/17; C12N 5/0783; A61P 35/00

(52) U.S. Cl. CPC A61K 35/17; A61P 35/00; C12N 5/0636

(57) ABSTRACT

The present invention relates to a method for preparing tumour-specific T cells, comprising the steps of: providing a tumour sample obtained from a patient, wherein the tumour sample comprises T cells that infiltrated the tumour; isolating T regulatory cells from the tumour sample, selecting at least one clonotype of the isolated T regulatory cells, determining the T cells receptor sequence of the selected clonotype, providing T cells other than T regulatory cells of patient, and transducing T cells other than T regulatory cells obtain from said patient with the determined TCR sequence of selected clonotype thereby yielding tumour specific T cells.

This application claims the benefit of priority of EP 19156745.2, filed 12. February 2019, which is incorporated herein by reference.

The present invention relates to a method for generating tumour specific T cells that counteract tumour immune escape.

### BACKGROUND

Concurrent to the great immunotherapeutic advances of cancer treatment in recent years, treatment failures caused by tumour escape have become an increasingly important issue. It has been shown that a major mechanism of tumour escape can be explained by the activity of CD8⁺ T-cells (cytotoxic T-lymphocytes, CTLs). While the initial tumour-destructive activity of CTLs is beneficial to the patient, under circumstances of incomplete tumour elimination, the CTLs' activity may lead to selective loss of cognate antigen from tumour cells in a process named immune editing, thus promoting tumour escape.

Whilst CD8⁺ T-cells specifically eliminate their tumour target cells, CD4⁺ regulatory T-cells (Tregs) show the opposite behaviour. Through their immunosuppressive activity, Tregs provide a growth advantage to tumour cells carrying the cognate antigen of these Tregs. In fact, Tregs are present throughout the progressing tumour and their number is often correlated with tumour size. This argues for the continuous presence of specific tumour antigens recognized by Tregs.

Given the persistence of specific antigens for Tregs in the tumour and in tumour derived metastases, it follows that the cognate T-cell receptors of Tregs (Treg TCRs) constitute prime tumour-specific recognition elements that may be employed for the development of immunotherapies counteracting tumour escape.

Based on this background, the problem underlying the present invention to provide means for the treatment of cancer that overcome T cell mediated tumour escape. A solution to this problem is provided by the subject matter of the independent claims, with particular embodiments discussed in the dependent claims and the following description.

### SUMMARY OF THE INVENTION

The fundamental concept underlying the present invention is the realization that regulatory T cells residing in the tumour, which mediate inhibition of cytotoxic T cells and other immune responses, may provide the key information, in form of their T cell receptor, to recognize tumour specific antigens. The potential of this concept can be turned into therapeutic tools in at least two ways:
Firstly, by isolating and transferring the genetic information encoding tumour-specific Treg TCRs into subsets of T-cells with anti-tumour capacity, particularly into Th1 cells. Such concept shall be referred to herein as “reverse immunosuppression”. Tumour-specific Treg TCRs must be selected for transduction to avoid the risk of autoimmunity.
Secondly, tumour-specific Treg TCRs can be used as probes to find the cognate antigen-peptide-MHCs, paving the way for the development of bispecific antibody or bispecific T cell Engager (BiTE® (Amgen Corp.)) related immunotherapies to counteract tumour escape.

The selection of tumour-specific Treg TCRs as provided by the invention represents a clear advantage for immunotherapeutic approaches. Two selection steps are employed in combination in order to achieve tumour specificity:
the frequency of Treg TCRs is compared between tumour and non tumour tissue. This will exclude autoreactive Treg TCRs of the tumour bearing tissue cells and Treg TCRs related to infections of the tumour bearing organ.
Treg TCRs are selected from induced Treg cells rather than from natural Treg cells. Induced Treg TCRs originate from tumour-specific T helper cells and thus, are expected to be safe for immunotherapeutic applications.

The preparations of tumour-specific T cells provided by the invention exhibit anti-tumour activity even in tumours with loss of function of Beta-2-Microglobulin through interactions with antigen presenting cells in the tumour microenvironment or more directly with malignant cells capable of MHC class II presentation.

### Terms and Definitions

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc.) and chemical methods.

The term virtually identical in the context of the present specification relates to a sequence that shows ≥98% identity in relation to the sequence it is said to be virtually identical to. The term virtually identical sequences is used for, but not limited to, sequences that originate from a sequencing event of the same physical sequence or an identical copy thereof, where methodical errors or misreads during the sequencing process have introduced artifactual errors into the sequence.

The term translation of nucleic acid sequences in the context of the present specification relates to the assignment of amino acid (polypeptide) sequences to nucleic acid sequences based on the genetic code and the known reading frame of the nucleic acid sequence. It does not relate to the biological act of translation in the cell performed by ribosomes.

The term CDR3 in the context of the present specification relates to the variable complementarity determining region 3 of the TCR complex. The size of CDR3 is characterized by the total number of amino acids (AA) and respective nucleotides from the conserved cysteine in the Vβ, or Vα or Vγ or Vδ segment to the position of the conserved phenylalanine in the Jβ or Jα, Jγ or Jδ segment.

The term “tumour sample” in the context of the present specification refers to a sample or a pool of samples obtained from a tumour of a patient. The tumour sample may also include or consist of a metastasis or a collection of metastases.

The term “non-tumour tissue sample” in the context of the present specification refers to a sample or a pool of samples obtained from non-tumour tissue comparable or identical to the tumour's tissue of origin or its position in case of metastasis. An advantageous source for such non tumour tissue sample is tissue in close proximity to the tumour of the patient.

In the present specification, the term positive, when used in the context of expression of a marker, refers to expression of an antigen assayed by a fluorescently labelled antibody, wherein the label's fluorescence on the structure (for example, a cell) referred to as “positive” is at least 30% higher (≥30%), particularly ≥50% or ≥80%, in median fluorescence intensity in comparison to staining with an isotype-matched fluorescently labelled antibody which does not specifically bind to the same target. Such expression of a marker is indicated by a superscript “plus” (⁺), following the name of the marker, e.g. CD4⁺. If the word “expression” is used herein in the context of “gene expression” or “expression of a marker or biomolecule” and no further qualification of “expression” is mentioned, this implies “positive expression” as defined above.

In the present specification, the term negative, when used in the context of expression of a marker, refers to expression of an antigen assayed by a fluorescently labelled antibody, wherein the median fluorescence intensity is less than 30% higher, particularly less than 15% higher, than the median fluorescence intensity of an isotype-matched antibody which does not specifically bind the same target. Such expression of a marker is indicated by a superscript minus (⁻), following the name of the marker, e.g. CD127⁻.

High expression of a marker, for example high expression of CD25, refers to the expression level of such marker in a clearly distinguishable cell population that is detected by FACS showing the highest fluorescence intensity per cell compared to the other populations characterized by a lower fluorescence intensity per cell. A high expression is indicated by superscript “high” or “hi” following the name of the marker, e.g. CD25^{high}. The term “is expressed highly” refers to the same feature.

Low expression of a marker, for example low expression of CD127, refers to the expression level of such marker in a clearly distinguishable cell population that is detected by FACS showing the lowest fluorescence intensity per cell compared to the other populations characterized by higher fluorescence intensity per cell. A low expression is indicated by superscript “low” or “lo” following the name of the marker, e.g. CD127^{low}. The term “is expressed lowly” refers to the same feature.

The expression of a marker may be assayed via techniques such as fluorescence microscopy, flow cytometry, FACS, ELISPOT, ELISA or multiplex analyses.

The term “clonotype” in the context of the present specification refers to a group of T cells that comprise T cell receptor nucleic acid sequences that exhibit a virtually identical nucleic acid sequence with respect to the variable region of the TCR, or that comprise T cell receptor amino acid sequences that exhibit a virtual identical amino acid sequence with respect to the variable region of the TCR. Clonotypes exhibiting a virtual identical amino acid sequence with respect to the variable region of the TCR may also referred to as clustertype.

### DETAILED DESCRIPTION OF THE INVENTION

The invention relates to a method for generating a preparation of tumour-specific T cells. This method comprises the following steps:
Sample collection step: A tumour sample comprising T cells that infiltrated the tumour is obtained from a patient. For the purpose of defining the invention, the actual step of obtaining the sample from the patient is not necessarily part of the method. It is important however that the sample is patient-specific and the general method disclosed herein is one that provides a preparation of T cells targeting one specific tumour by reversing the immunosuppression mediated by certain regulatory T cells residing in that tumour.
Tumour T cell isolation step: T regulatory cells are isolated from the tumour sample, yielding a preparation of tumour infiltrating Treg (tiTreg) cells. Methods are known in the art to generate a T cell preparation from tissue (GentieMACS® and MicroBeads, Miltenyi Biotec).
Tumour T cell sequencing step: A plurality of nucleic acid sequences encoding tumour infiltrating T cell receptors is obtained from the T regulatory cells obtained in the previous step, yielding a set of tumour TCR sequences. Ideally, this set of sequences represents each T cell comprised in the sample. The tumour T cell derived nucleic acid need to be sufficient for subsequent determination of both a short CDR3 sequence tract for clonotyping, and the subsequent generation of the full TCR alpha and beta chains for constructing the transgene TCR expression cassettes that are transferred into recipient T cells.
Sequence selection step: one or several nucleic acid sequences encoding a tumour-infiltrating T regulatory T cell receptor (tiTreg TCR) are selected, yielding one or several selected tiTreg TCR sequences. The selected tiTreg TCR sequences are specific for the tumour. Different methods exist for determining which tiTreg TCR sequences have this specificity, including but not limited to the use of the preferred localization of a unique regulatory T cell clone in the tumour as determined quantitatively by the ratio of the clonotype frequencies between tumour and non-tumour tissue (particularly: non-tumour tissue adjacent to the tumour site) as an indicator of tumour specificity and to the comparison of tumour derived regulatory T cell receptor sequences with data bases.
Recipient T cells: recipient T cells that are not T regulatory cells are obtained from the patient. Again, for the purpose of defining the invention, the actual step of obtaining the cells from the patient is not necessarily part of the method. In most embodiments, the recipient T cells are helper cells with a small proportion of naïve T cells and the majority of them exhibiting a central memory and an effector memory phenotype, representative of the peripheral blood T cell repertoire. In certain embodiments, the use of enriched naïve CD4⁺ T cells as recipient cells may be advantageous.
Gene transfer step: A nucleic acid encoding the selected tiTreg TCR under control of a promoter sequence operable in the recipient T cells is transferred into the recipient T cells, thereby yielding the preparation of tumour-specific T cells according to the invention. For the purpose of the invention, the most feasible method of transgene transfer is deemed to be transduction, also known as viral DNA transfer. A number of technologies have been demonstrated to work and several tested gene transfer vehicles are available for clinical use, namely retroviral, particularly lentiviral gene transfer (see Voss et al., Adoptive Immunotherapy: Methods and Protocols, Springer Science and Business Media, 2005). Transfection, by way of non-viral/plasmid DNA transfer may be an alternative.

Obtaining a plurality of nucleic acid sequences encoding T cell receptors from the isolated T regulatory cells is equivalent to analysing the T cell receptor repertoire of the cells. In other words, an estimation is made as to which TCR sequences are present in the T cell population, and at what abundance (what is the relative incidence among the total number of TCR sequences).

Prior to the tumour T cell sequencing step, CD4⁺ cells are separated from the T cells isolated from the tumour sample, or the tumour infiltrating T cells separated from the tumour are restricted to CD4⁺ cells in the first place, for example by selection through CD4 antibodies. In the tumour T cell sequencing step, only or predominantly sequences encoding T cell receptors originating from CD4⁺ T cells are sequenced.

Non-limiting options for conducting the T cell sorting for analysing Treg include a) using cell surface marker CD4⁺CD25⁺CD127^{low}/−, b) using CD4⁺ and intracellular FoxP3⁺ staining, c) using surface marker CD4⁺CTLA4^{high} or CD4+CD39^{high}. For cell sorting of non-Treg conventional CD4 T cells (Tconv) the surface marker CD4⁺CD25⁻CD127^{high} can be used to determine potential overlaps in TCR-repertoires of Treg and Tconv. Separated T cell subsets are selected for DNA extraction and α- and/or β-TCR/CDR3 NGS sequence analysis is performed.

In particular embodiments, T cells that are characterized by expression of CD4 and CD25, and low expression of CD127 (CD4⁺CD25⁺CD127^{low}) and/or by expression of both CD4 and FOXP3 (CD4⁺FOXP3⁺) are isolated in the tumour T cell isolation step, and subsequently sequenced exclusively. In certain embodiments, T cells with a T regulatory phenotype are characterized by markers known in the art including, without being limited to, CTLA-4, TIM3, GITR, LAG-3, CD69, TGF-beta, IL-10, particularly characterized by expression of CD4 and CD25 (Mason et al. J. Immunol. 2015, 195 (5) 2030-2037).

In particular embodiments, the sequence selection step comprises comparing said tumour TCR sequences among each other, thereby determining the relative incidence (frequency) of any individual sequence, and selecting sequences on account of their incidence in the tumour and in non-tumour tissue or blood. This information is valuable in determining the contribution of a particular sequence to the totality of the TCR mediated immunological equilibrium, and the likelihood of it being a representative of an important population of T cells, in the sense that they are likely to be specific for an antigen that is present within the local tumour tissues. The tumour-specific clonotype is absent or exhibits a frequency of <20%, <15%, <10% or <5% within the set of non-tumour tissue CD4⁺ TCR sequences. Conversely, if the ratio between the frequency of a particular clonotype in the tumour and the frequency of the same clonotype in non-tumour tissue is a 5 this is indicative of tumour specificity.

In particular embodiments, the sequence selection step comprises comparing said tumour TCR sequences to a set of TCR sequences obtained from T cells (particularly to a set of TCR sequences obtained from CD4⁺ T cells, more particularly CD4⁺CD25⁺ T cells, even more particularly CD4⁺CD25⁺CD127^{low} and/or CD4⁺FOXP3⁺ T cells) obtained from a non-tumour tissue sample of the same patient. By comparing the prevalence of particular TCR sequences between these two sites or samples, it is possible to determine whether a TCR sequence or a set of TCR sequences is specific for the tumour, or representative of TCR sequences residing outside of the tumour environment as it is the case in tissue specific autoimmunity and/or infection. The invention relies on a method of distinguishing tumour-specific from non-tumour regulatory TCR sequences, the latter being potentially harmful if isolated and used as transgene TCR sequences in an immune-stimulatory context as a cancer treatment, but the former bearing the potential to guide an anti-tumour response.

Additional information such as the existence of ubiquitous, systemically present clonotypes can be obtained by comparing the tumour derived Treg TCR sequences to those derived from blood CD4⁺ cells.

In particular embodiments, the sequence selection step comprises comparing said tumour TCR sequences to a set of reference TCR sequences. Conceivably, recurrent practice of the invention disclosed herein will lead to a wealth of TCR information enabling the creation of a database of tumour and non-tumour TCR sequences which are common between patients, allowing the user to identify tumour-specific TCR sequences without reference to a second biological sample.

Sequences are selected that are characterized by high incidence (frequent) in the tumour and/or unique to the set of tumour TCR sequences.

In particular embodiments, prior to the sequence selection step, nucleic acid sequences are translated to amino acid sequences and the comparison of tumour TCR sequences is performed on the basis of amino acid sequences. The skilled person recognizes that any of the sequence determination and comparison steps disclosed herein can be performed on the nucleic acid sequence level, but also-often in a second step-on the amino acid sequence level. While obtaining sequences from samples is almost invariably going to be performed on the nucleic acid level, sequence alignment and comparison may be performed on the amino acid level as certain differences in nucleic acid sequence may not lead to a difference in the polypeptide, which for the purposes of antigen or MHC recognition is the operative entity on the biological level.

In particular embodiments, the sequence selection step comprises the steps of
aligning the set of tumour TCR sequences in an alignment step;
grouping tumour TCR sequences aligned in the alignment step into a plurality of tumour sample clonotypes, wherein sequences comprised within a particular clonotype exhibit a virtually identical sequence or an identical sequence,
determining the number of tumour TCR sequences associated with each clonotype, thereby yielding a clonotype frequency within the set for each of said clonotypes;
selecting a tumour-specific clonotype from said plurality of tumour sample clonotypes, wherein said tumour-specific clonotype is one of the 100 most frequent clonotypes, particularly one of the most 50, 20, 10 or even only 5 of said plurality of tumour sample clonotypes.

Particularly, a first T cell receptor nucleic acid sequence is virtually identical to a second T cell receptor nucleic acid sequence, if both sequences differ in not more than one base-pair position.

In certain embodiments, a first T cell receptor nucleic acid sequence is virtually identical to a second T cell receptor nucleic acid sequence, if both sequences differ in not more than one base pair, and the first T cell receptor nucleic acid sequence exhibits in the respective sample, particularly in the tumour sample, an at least twentyfold frequency compared to the second T cell receptor nucleic acid sequence. Consequently, both first and second T cell receptor nucleic acid sequences are assigned to the same clonotype.

Likewise, a first T cell receptor amino acid sequence is virtually identical to a second T cell receptor amino acid sequence if both amino acid sequences differ from each other in not more than at one or two positions. The above-mentioned T cell receptor amino acid sequences may be comprised within the alpha and/or beta chain of the TCRα/β or within the gamma or delta chain of the TCRγ/δ.

The clonotype frequency is a measure of the relative or absolute frequency of the T cell identified by the TCR nucleic acid sequence within the set of sequences for which the frequency is determined.

Particularly, the clonotype frequency in a given sample is a measure of the relative or absolute frequency of the T cell identified by the TCR nucleic acid sequence within said sample.

In certain embodiments, the T cell receptor nucleic acid sequences of the above-mentioned plurality are comprised within nucleic acid sequences encoding one of the polypeptide chains that form a human T cell receptor, particularly TCRα/β or TCRγ/δ. In certain embodiments, the T cell receptor nucleic acid sequences of the above-mentioned plurality do not comprise non-coding nucleic acid sequences. Non-coding sequences refer to clonotypes with stop-codons or frame shifts that lead to non-functional TCR protein sequences.

In certain embodiments, a tumour-specific T cell receptor nucleic acid sequence is characterized by a length of 30 nucleotides to 110 nucleotides.

In certain embodiments, the tumour-specific T cell receptor nucleic acid sequence encodes a unique amino acid sequence comprised within any one of the polypeptide chains (alpha, beta, gamma and delta) that form a human T cell receptor, wherein the unique amino acid sequence exclusively occurs in a particular clonotype or clustertype and not any other clonotype or clustertype.

In particular embodiments, comparison of the sequences is performed with respect to the CDR3 sequence tract and all members of a particular clonotype exhibit an identical CDR3 sequence.

In particular embodiments, the method further comprises the following steps that enable to obtain and identify a set of non-tumour sequences:
A non-tumour tissue sample is obtained from said patient (as laid out before, not necessarily a technical part of the method as claimed herein; the sample may have been obtained previously);
A nucleic acid preparation is isolated from the non-tumour sample in a nucleic acid isolation step;
a plurality of non-tumour tissue T cell receptor sequences is obtained from the non-tumour tissue sample; this yields a set of non-tumour tissue TCR sequences.
the plurality of non-tumour tissue T cell receptor sequences is aligned;
the non-tumour tissue T cell receptor sequences are grouped into a plurality of non-tumour tissue clonotypes. T cell receptor sequences grouped into a particular clonotype exhibit a virtually identical or an identical sequence;
the number of non-tumour tissue T cell receptor sequences associated with each clonotype is determined, thereby yielding a clonotype frequency within their respective set for each of the clonotypes;
a tumour-specific clonotype is selected from the plurality of tumour sample clonotypes. The tumour-specific clonotype is absent or exhibits a frequency of <20%, <15%, <10% or <5% within the set of non-tumour tissue TCR sequences.

In particular embodiments, obtaining T cell receptor sequences comprises the steps of
isolating T cells from the tumour sample, non-tumour tissue sample and blood sample and isolating nucleic acid from the isolated T cells, and
conducting a nucleic acid amplification reaction that specifically amplifies T cell receptor nucleic acid sequences.

Methods for obtaining TCR sequences are known in the art. WO2014096394, also published as US2015337368, both incorporated by reference herein in their entirety, show methods used in the examples. Another system is available under the tradename “ImmunoSEQ” from Adaptive Biotechnologies world-wide.

In particular embodiments, the nucleic acid amplification reaction specifically amplifies a sequence encoding the CDR3 region of a chain of the human T cell receptor, particularly the CDR3 region of the alpha chain or the beta chain of the human T cell receptor.

While the step of selecting tiTreg sequences does not require knowledge about the composition of the associated full T cell receptor with respect to individual alpha chains and beta chains, the reconstruction of a functioning TCR encoding expression vector for transfer into the recipient cell does require this knowledge. Several strategies may be employed to achieve this.

The currently practiced methodology is to obtain TCR sequences twice in the process: firstly, a first portion of T cells obtained from the tumour sample are sequenced only with regard to the CRD3 sequence tract. This allows determination of clonotypes. The non-tumour sample is sequenced similarly.

Then, in a second sequencing step, a second portion of T cells obtained from the tumour sample are separated into single cells and sequenced individually. This sequencing round (step) affords information about the full TCR polypeptide chains expressed in any one single cell, and enables construction of the respective expression vectors for transfer into the transfer system employed in the gene transfer step. One technology that can be employed is 10× sequencing from 10× genomics Inc., described in U.S. Pat. Nos. 9,644,204, 9,975,122, 10,053,723 and 10,071,377, all of which are incorporated by reference herein.

In particular embodiments, recipient T cells are selected from the group comprising cytotoxic T cells and T helper cells. Herein, the method comprises a step of selecting T helper cells from a sample obtained from the patient, yielding an enriched T helper cell preparation. The recipient T cells are prepared by depletion of CD8⁺ T cells and CD4⁺ regulatory T cells, particularly from a lymphocyte preparation of the patient.

In certain embodiments, the recipient T cells (which are not regulatory cells) belong to an enriched fraction of T helper cells described as CD3⁺CD4⁺CD8⁻CD25⁻ that is isolated from PBMCs of the same patient by e.g. CD8 and CD25 negative selection.

In certain embodiments, the recipient T cells are part of an enriched fraction of T helper cells described as CD3⁺CD4⁺CD8⁻CD127⁺ that is isolated from PBMCs of the same patient by CD8 negative and CD127 positive selection.

The enriched T cell preparation is subjected to the gene transfer step.

Alternatively, cytotoxic T cells are selected from a sample obtained from the patient, yielding an enriched cytotoxic T cell preparation.

The term “non-tumour tissue sample” in the context of the present specification refers to a sample or a pool of samples obtained from tissue of the same type as the tissue in which the tumour originates; one non-limiting example for a source of non-tumour tissue is tissue in close proximity to the tumour of a patient.

Particularly, the non-tumour tissue T cell receptor amino acid sequences are grouped into the plurality of non-tumour-tissue clonotypes in the same manner as the T cell receptor amino acid sequences obtained from the tumour into the plurality of tumour sample clonotypes, particularly to allow a re-assignment of a clonotype identified in a tumour sample to a non-tumour tissue clonotype.

Particularly, a clonotype can be assigned to a non-tumour clonotype, if any one of the T cell receptor amino acid sequences of the plurality of T cell receptor amino acid sequences comprised within this clonotype is virtually identical or identical to a T cell receptor amino acid sequence comprised within a non-tumour clonotype.

Particularly, a clonotype identified in a tumour can be assigned to a non-tumour clonotype, if
a) any one of the T cell receptor nucleic acid sequences of the plurality of T cell receptor nucleic acid sequences comprised within this clonotype is virtually identical to a T cell receptor nucleic acid sequence comprised within a non-tumour clonotype and/or
b) a T cell receptor amino acid sequence encoded by a T cell receptor nucleic acid sequence of the plurality of T cell receptor nucleic acid sequences comprised within this clonotype is identical to a T cell receptor amino acid sequence encoded by a T cell receptor nucleic acid sequence comprised within the non-tumour sample clonotype.

In certain embodiments, the non-tumour sample is a sample of non-tumour tissue of the same type of tissue as the tumour, particularly a sample of non-tumour tissue adjacent to the tumour. Such non-tumour tissue can be identified by common techniques such as ultra sound examination, radiography, CT or immunostaining.

The term “blood sample” or “sample from blood” in the context of the present specification refers to a sample from blood or a pool of samples obtained from blood of a patient.

In certain embodiments, the T cell receptor amino acid sequences obtained from the blood sample are grouped into the plurality of blood sample clonotypes in the same manner as the T cell receptor amino acid sequences obtained from the tumour into the plurality of tumour sample clonotypes, particularly to allow an assignment of a tumour sample clonotype to a blood sample clonotype.

Particularly, a tumour-specific clonotype can be assigned to a blood sample clonotype if any one of the T cell receptor amino acid sequences of the plurality of T cell receptor amino acid sequences comprised within this clonotype exhibits a virtually identical or identical sequence to a T cell receptor amino acid sequence comprised within a blood sample clonotype.

In certain embodiments, the selected tumour-specific clonotype cannot be assigned to a known clonotype being reactive to viruses such as the human cytomegalovirus or the Epstein-Barr-virus.

Such assignment may be performed by bioinformatics methods, wherein a tumour-specific T cell receptor nucleic acid sequence comprised within the selected tumour-specific clonotype is compared to nucleic acid sequences of known clonotypes being reactive to viruses such as the human cytomegalovirus or the Epstein-Barr-virus.

Particularly, the selected tumour-specific clonotype cannot be assigned to a clonotype known to be reactive to viruses such as the human cytomegalovirus or the Epstein-Barr-virus, if
a) none of the T cell receptor nucleic acid sequences of the plurality of T cell nucleic acid sequences comprised within this clonotype is virtually identical to a T cell receptor nucleic acid sequence comprised within the known clonotype,
b) none of the T cell receptor amino acid sequences encoded by a T cell receptor nucleic acid sequence of the plurality of T cell receptor nucleic acid sequences comprised with this clonotype is identical to a T cell receptor amino acid sequence encoded by a T cell receptor nucleic acid sequence comprised within the known clonotype, or
c) none of the T cell receptor amino acid sequences of the plurality of T cell amino acid sequences comprised within this clonotype is virtually identical or identical to a T cell receptor amino acid sequence comprised within the known clonotype.

In certain embodiments, the nucleic acid isolation step comprises the steps of
a. isolating T cells from said tumour sample and isolating nucleic acid from said isolated T cells, and/or
b. conducting a nucleic acid amplification reaction that specifically amplifies T cell receptor nucleic acid sequences.

In certain embodiments, the tumour-specific T cell receptor nucleic acid sequence encodes the CDR3 region of a chain of the human T cell receptor, particularly the CDR3 region of the alpha chain or the beta chain of the human T cell receptor or the tumour-specific T cell receptor amino acid sequence is comprised within the CDR3 region of a chain of the human T cell receptor, particularly the CDR3 region of the alpha chain or the beta chain of the human T cell receptor.

In certain embodiments, the tumour-specific nucleic acid sequence is comprised within an RNA, particularly encoding an amino acid sequence comprised within the CDR3 region of the alpha chain or the beta chain of the human T cell receptor.

In certain embodiments, the tumour-specific nucleic acid sequence is comprised within an RNA, particularly encoding amino acid sequences comprised within the CDR3 regions of both alpha chain and beta chain of the human T cell receptor.

In certain embodiments, the tumour-specific nucleic acid sequence is comprised within an RNA, particularly encoding long stretches of amino acid sequences of the alpha chain or the beta chain of the human T cell receptor.

Particularly, the tumour reactivity of the tumour-specific Treg TCR-transduced T cell preparation of the invention may be confirmed by:
co-culturing at least an aliquot of the above mentioned Treg TCR-transduced tumour specific T cell preparation of the invention with a cell suspension of the autologous tumour of the patient (“target cell suspension”) which is either untreated or pre-treated with interferon gamma to stimulate expression of MHC class II molecules, or a lysate of the autologous tumour together with autologous antigen presenting cells of the patient, and
determining the amount of interferon gamma or TNF alpha produced by the T cell preparation in presence of the target cell suspension, and/or determining the level of a T cell activation marker in the T cell preparation in presence of the target cell suspension, particularly determining the level of OX40, CD107a, CD137, CD154, LAG3, PD-1, B7-H4, PD-1, CD39, a member of Butyrophilin, a Butyrophilin-like protein and/or CD69.

In one embodiment, the method of the invention provides a preparation of tumour-specific T cells by the following steps:
CD4⁺ T cells are isolated from a tumour sample obtained from a patient, the T cell receptor sequences for a representative set of the isolated tumour infiltrating (TIL) CD4⁺ T cells are determined and grouped into tumour infiltrating (TIL) CD4⁺ T cell clonotypes; the frequency of each clonotype among all TIL CD4⁺ cells is determined.
Likewise, CD4⁺ T cells are isolated from a non-tumour sample obtained from the same patient, the T cell receptor sequences for a representative set of the isolated non-tumour CD4⁺ T cells are determined and grouped into clonotypes; the frequency of each clonotype among all non-tumour CD4⁺ cells is determined.
T cell receptor sequences are determined for a representative set of tumour infiltrating CD4⁺ regulatory (CD25⁺ CD127^{lo} and/or FOXP3⁺) T cells. Again, the clonotype distribution and the frequency of the clonotype (occurrence of the clonotype among all TCR sequences determined for the set of TIL CD4 regulatory cells) is determined.
T cell receptor sequences are determined for a representative set of tumour infiltrating CD4⁺ conventional (CD25⁻ CD127⁺ and/or FOXP3⁻) cells. Again, the clonotype distribution and the frequency of the clonotype (occurrence of the clonotype among all TCR sequences determined for the set of TIL CD4 conventional cells) is determined.
In the selection step, one or several TCR sequences encoding a tumour-infiltrating T regulatory T cell receptor (tiTreg TCR) are selected, yielding one or several selected tiTreg TCR sequences. The selection of a clonotype can be made applying the following criteria: The frequency of a clonotype among TIL CD4+ cells is significantly higher (in certain embodiments: at least 5 times higher) than the frequency of the clonotype among non-tumour CD4⁺ cells, and the clonotype is characterized as an induced Treg, i.e. it exhibits an overlap in the TCRsafe analysis between the tiTreg (CD4⁺CD25^{high}CD127^{low} or CD4⁺FOXP3⁺) and tiTconv (CD4⁺CD25^{low}CD127⁺ or CD4⁺FOXP3⁻) fractions, and/or the 10× Genomix single cell analysis detects in TIL CD4⁺ cells belonging to the clonotype both FOXP3 expressing and FOXP3 negative cells.
In a gene transfer step, one or more nucleic acid expression vectors encoding tiTreg TCR selected in the previous step are transferred into recipient T cells under control of a promoter sequence operable in the recipient T cells, thereby yielding a preparation of tumour-specific T cells.

In certain embodiments, the gene transfer step is preceded by a transgene generating step, wherein the nucleic acid encoding said selected tiTreg TCR under control of a promoter sequence is generated by
a. separating a plurality of T regulatory cells obtained from said tumour sample into single cells;
c. obtaining a plurality of complete TCR sequence sets, each TCR sequence set comprising a full TCR alpha and TCR beta polypeptide sequence from said plurality of cells;
d. assigning the selected tiTreg TCR sequence to a complete TCR sequence set, yielding a complete tiTreg TCR sequence set;
e. inserting sequences encoding the full TCR alpha and TCR beta polypeptide sequence of said complete tiTreg TCR sequence set determined in the preceding step into a gene expression construct.

Another aspect of the invention relates to a preparation of T cells obtained by the method of the invention, particularly for use in treatment of cancer, or in prevention of its recurrence.

Yet another aspect of the invention relates to a method to identify tumour-specific neoantigens by employing tumour-specific Treg TCRs identified and isolated as described in the above aspects and embodiments of the invention. These neoantigens may be employed as tumour vaccines or in the development of bispecific antibody or bispecific T cell Engager (BiTE® (Amgen Corp.)) related immunotherapies to counteract tumour escape.

Major targets of anti-tumour T cell responses are so-called neoantigens resulting from tumour-specific somatic mutations. They are identified by whole-exome- and transcriptome sequencing using genomic DNA and RNA of tumour cells derived from biopsies or surgical resection material. For comparison, corresponding nucleic acids from tumour-adjacent normal tissue or peripheral blood lymphocytes are sequenced as well. Neoantigen candidates, especially those with mutations in common oncogenic driver genes like KRAS, NRAS, TP53, PIK3CA, EGFR, BRAF, and/or the like are then tested for recognition by Treg TCR-transduced T cells in vitro. In addition to mutated neoantigens, candidate tumour-associated antigens (TAA) such as tumour type-specific cancer-germline antigens or antigens overexpressed or aberrantly (in alternative reading frames) expressed in tumour cells can be identified by transcriptome sequencing as well and tested for recognition by Treg TCR-transduced T cells. Both, peptides derived from neoantigens containing the amino acids exchanged by mutations and long-overlapping peptide libraries spanning the complete amino acid sequences of candidate TAA, are synthesized, pulsed onto autologous or HLA-matched antigen-presenting cells and tested for recognition by Treg TCR-transduced T cells via functional assays like IFN-γ-Elispot assays, ELISA or comparable tests.

Thus, in one embodiment the invention relates to a method for determining the ability of a neoantigen to elicit tumour-specific T cell responses. This method comprises the steps of:
Contacting a neoantigen candidate oligopeptide or neoantigen polypeptide with antigen-presenting cells derived of a patient, or with antigen presenting cell lines presenting HLA molecules representative of the HLA repertoire of a patient; or
Contacting candidate oligopeptides or polypeptides derived from unmutated antigens with tumour-restricted expression with antigen-presenting cells derived of a patient, or with antigen presenting cell lines presenting HLA molecules representative of the HLA repertoire of a patient;
Contacting the antigen presenting cells with T cells (non-Treg TC) expressing the tumour-specific TCR sequences identified according to the first aspect of the invention, and
Determining whether the interaction between tumour antigen-candidate-presenting antigen presenting cell and the transduced T cell leads to the T cell's activation. If activation is registered, the neoantigen candidate is assigned a neoantigen status.

Likewise, unmutated antigen candidates with tumour-restricted expression recognized by the tiTreg-transgenic T cells are assigned tumor-associated antigens.

The invention further provides the use of the tumour-specific regulatory TCR sequences as a biomarker.

The nucleotide sequences of Treg TCRs may be synthesized and transferred into expression vectors (by way of non-limiting example, retroviral expression vectors) for the purpose of transducing fresh effector T cells. The sequences of the α- and β-chains may be designed as codon-optimized sequences (or as a nucleic acid sequence significantly differing from the TCR sequence found in the patient without being different on the amino acid level), giving them unique sequence features that can easily be recognized and quantified by high throughput sequencing of ex vivo material such as peripheral blood lymphocytes. After adoptive transfer of Treg TCR-transduced T cells into the patient, the Treg TCR-sequences can be monitored at different time points after therapy and correlated with the clinical course of the treated patient.

The invention is further illustrated by the following examples and figures, from which further embodiments and advantages can be drawn. These examples are meant to illustrate the invention but not to limit its scope.

### DESCRIPTION OF THE FIGURES

FIG. 1 shows a flow chart of one embodiment of the method of the invention.

FIG. 2 shows a flow chart depicting the principles of the method of the invention.

FIG. 3 shows the results of a TCRβ sequencing run for one tumour patient (V84/13). Each line corresponds to one unique βTCR/CDR3, which is found in a unique type of T cells (clonotype). The columns from left to right denote: a. the relative frequency of that type of T cell, b. the frequency given in number of sequencing reads, c. the V-segment ID, d. the J-segment ID, e. the CDR3 peptide, f. the sequenced region as nucleic acid sequence covering the complete CDR3.

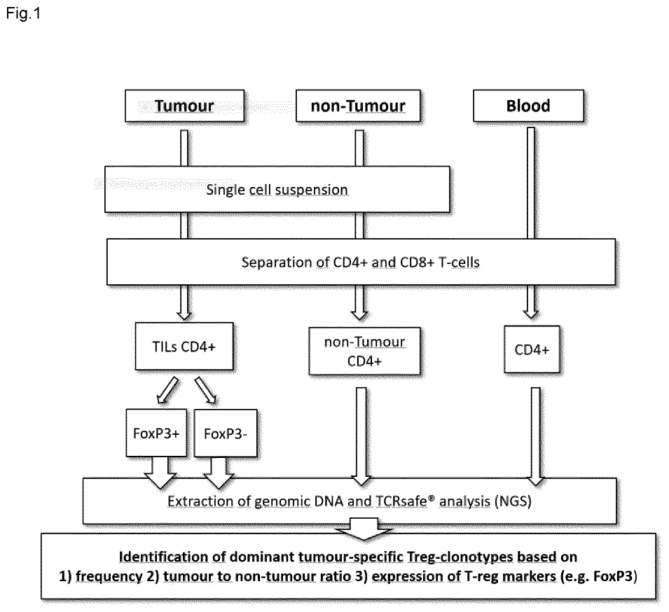

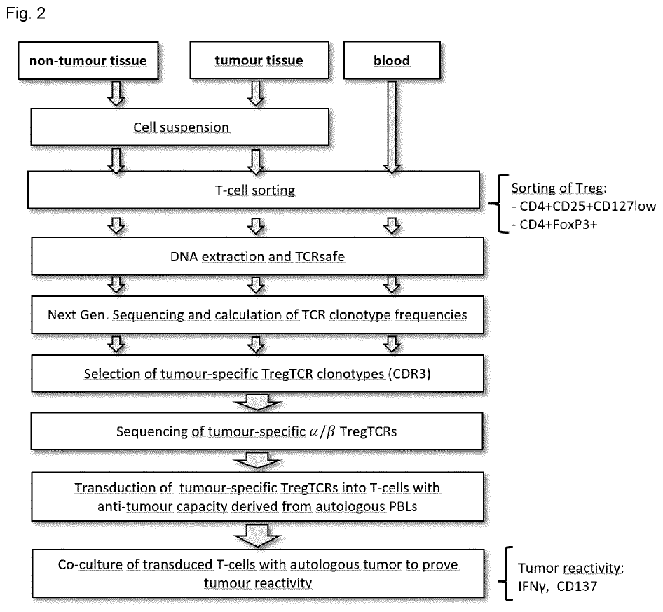

FIG. 4 shows the assignment of induced tumour-specific Treg TCR sequences in a patient. All samples are from one patient. The table depicts the CDR3s peptide, V-segment and J-segment of each of the TOP 30 CD4⁺TIL clonotypes. Sorting is with respect to TIL CD4⁺ clonotypes (column F). Dark grey shaded numbers in column I indicate tumour specificity as determined by a 5-fold or higher enrichment of the unique clonotype in tumour as compared to adjacent non tumour tissue. Sorting with Tconv and Treg markers are shown in columns G and H, respectively. The 10× Analysis is shown in column J. Sorting identifies induced Treg clonotypes in rows 10 and 26. These clonotypes exhibit an overlap between the regulatory and conventional T cell fractions. The 10× analysis, which includes Treg characterization and α/β-pairing of TCRs in one go, confirms the induced Treg nature of clonotypes in rows 10 and 26. Furthermore, the 10× analysis identifies the clonotype in row 25 as a natural Treg. The clonotype in row 21 can be interpreted as conventional Th cell, since the 10× analysis did not show any FOXP3 signal.
The frequency values given in columns D, E, F, G and H are given in percent of sequences in relation to all sequences of the class. As an example, the frequency of the CDR3 peptide assigned SEQ ID NO 37 among non-tumour CD4 cells is 0,2%.

FIG. 5 shows the assignment of induced tumour-specific Treg TCR sequences in the same patient as FIG. 4. All samples are from one patient. The table shows the CDR3beta peptide, V-segment and J-segment of each of the TOP 20 tumour-specific TIL Treg clonotypes enriched by cell sorting with Treg markers. The table is sorted with respect to column G. The results of the 10× analysis are depicted in column I, classifying the cells in natural and induced regulatory T-cells or unspecified CD4⁺ T-cells. The 10× analysis comprises Treg characterization and α/β-pairing of TCRs in one go.

FIG. 6 shows another example of assignment of induced tumour-specific Treg TCR sequences. Samples (blood, tumour, non-tumour) were obtained from a different patient as the patient providing the samples on which the data of FIGS. 4 and 5 are based. The ratio between TILs (CD4⁺) and T-cell clones selected from non-Tumor is filtered for values >5, which indicates tumour specificity. It is evident that clonotypes (row 1 and 16) are of significant frequency in the tumour and were detected as induced Tregs by FOXP3 expression analysis with 10×Genomics single cell sequencing technology. In addition, among the top 20 TIL clonotypes there were 3 natural Tregs (row 13, 15, 17) identified by 10×Genomics analysis. The Treg CloneID denotes the group of induced Treg clones with FOXP3 positive and FOXP3 negative cells sharing the same CDR3 region. For these clones the 10×Genomics analysis delivered both chains of the T-cell receptor.

FIG. 7 shows the complete T cell receptor CDR3 sequences (Beta- and Alpha-chains) for the 3 examples of induced or natural Tregs depicted in FIG. 4.

### EXAMPLES

Handling and processing of different tissues and cell sorting
a) Tumour samples are either defined as one sample or a set of replicate samples taken from tumour tissue. In practice, the material to analyse the TCRs is obtained by either
   i.) Selecting distinct biopsies or different areas of one biopsy. This may be assisted by immunohistochemical staining, wherein particularly Treg cells are immune-histologically stained with Treg markers such as CD4, FOXP3, CD25, CD45RA, HLA-DR, CTLA4, CD39, and stained regions are selected for DNA extraction.
   ii.) Lysis of tumour tissue e.g. by bead-based technologies for preparation of single cell suspensions as starting point for TCR analysis. There are several options of T cell sorting for analysing Treg. These include a) using cell surface marker CD4⁺CD25⁺CD127^{low/-}, b) using CD4⁺ and intracellular FOXP3⁺ staining, c) using surface marker CD4⁺CTLA4^{high} or CD4⁺CD39^{high}.

For cell sorting of non-Treg conventional CD4 T cells (Tconv) the surface marker CD4⁺CD25⁻ CD127^{high} and/or CD4⁺FOXP3⁻ can be used to determine potential overlaps in TCR-repertoires of Treg and Tconv. Such an overlap of a unique clonotype in the Treg and Tconv fraction is an indication of induced Treg formation. When using Treg TCRs for immunotherapeutic approaches, it can make a difference whether the TCR is derived from a natural or an induced regulatory T-cell. The TCR of an induced Treg originates from a tumour-specific T helper cell. Thus its use should be tumor-specific and safe. On the other hand, TCRs of natural regulatory T-cells may recognize cognate self-antigens at other sites in the organism, causing adverse effects by induction of autoimmunity. Such side effects may be of inflammatory rather than cytotoxic nature and may require anti-inflammatory treatment Moreover, peripherally induced Treg cells can be distinguished from thymic natural Treg cells by lack of expression of the surface marker neuropilin1 and the intracellular marker Helios. Separated T cell subsets are selected for DNA extraction and α- and/or β-TCR/CDR3 NGS sequence analysis is performed.

In addition, tumour samples may be stored under cell-preserving conditions as resource for cell material.
b) Non-tumour samples from the same patient are selected from tissue/regions adjacent to tumour sample, if possible in replicates, whenever possible from distinct tissue spots. Tissue samples are taken or single cell suspensions are prepared. There are several options of T cell sorting for identifying Treg. These include a) using the cell surface marker CD4⁺CD25⁺CD127^{low/-}, b) using CD4⁺ and intracellular FOXP3⁺ staining, c) using surface marker CD4⁺CTLA4^{high} or CD4⁺CD39^{high}. For cell sorting of non-Treg conventional CD4 T cells (Tconv) the surface marker CD4⁺CD25⁻CD127^{high} can be used to determine potential overlaps in TCR-repertoires of Treg and Tconv. Tissue samples or separated T cell subsets are selected for DNA extraction and α- and/or β-TCR/CDR3 NGS sequence analysis is performed.

Blood samples are taken from the same patient: By standard hematologic fractionation cellular components are isolated from full blood. Cellular components may be separated in different T cell subsets. After DNA extraction, α- and/or β-TCR/CDR3 NGS sequence analysis is performed.

Providing Anti-Tumour T-Cells by Gene Transfer of Tumour-Specific Treg TCRs

Preparation of Cell Suspensions of T-Lymphocytes from Tumour (NSCLC) and Lung Tissue:

Each tumour specimen is dissected free of surrounding normal tissue and necrotic areas. Approximately 0.8 g cubes from tumour and normal lung tissue are cut into small chunks measuring about 2-3 mm in each dimension. Sliced tumour (and also non-tumour) biopsies are subjected to a commercial mechanical/enzymatic tissue dissociation system (gentleMACS, Miltenyi Biotec), using the Tumour Dissociation Kit (Miltenyi Biotec) and following the manufacturers instructions.

After gentleMACS disaggregation, cell suspensions are passed through 70-μm strainers. Aliquots of tumour cells are taken at this point and cryopreserved in 10% DMSO (Sigma-Aldrich) and 90% FCS (Life Technologies) for later use. The remaining cell suspension is subjected to density gradient centrifugation using a 40%/80% step gradient of Percoll® (GE Healthcare Europe GmbH) in PBS/RPMI 1640. T-lymphocytes are harvested from the interphase and washed in complete medium (RPMI 1640, Lonza). Subsequently, T-lymphocytes are cultured by placing cells at a concentration of 0.5×10⁶ cells/ml in each well of a 24-well tissue culture plate with 2 mL of TexMACS medium (=recovery medium, Miltenyi Biotec) supplemented with 100 IU/mL penicillin, 100 μg/mL streptomycin. Plates are placed in a humidified 37° C. incubator with 5% CO₂ and cultured overnight.

Sorting of T-Lymphocytes

The next day, cells are harvested and pooled from the wells. For isolation of the Treg fraction (CD4⁺, CD25^{high}, CD127^{low/neg}) and the corresponding Tconv fraction (CD4⁺, CD25^{low/neg}, CD127⁺) cells are stained for FACS sorting with the following antibody panel (Miltenyi Biotec): human CD3-APC-Vio770, CD4-PE-Vio770, CD25-PE, CD127-APC. Additionally cells are stained to test for viability using 7-Aminoactinomycin D (7-AAD, BioLegend). DNA is extracted from each separated fraction and utilized for library preparation.

Genomic DNA Isolation and TCRsafe® Analysis

Genomic DNA (gDNA) is extracted from tissue materials using the NucleoSpin® Tissue Kit from Macherey-Nagel (Düren, Germany). Blood gDNA is isolated from 2-3 ml fresh blood with either QIAamp® DNA Blood Mini Kit (Qiagen, Hilden, Germany) or AllPrep® DNA/RNA/miRNA Universal Kit (Qiagen) following the manufacturer's protocols.

CDR3 regions of the TCRβ-chain are sequenced with NGS (Illumina MiSEQ) technology following the TCRsafe® method, a proprietary 2-step PCR amplification procedure (as disclosed in WO 2014/096394 A1) which uses TCRβ primers binding specifically to the V- and J-segments adjacent to the CDR3 region. Genomic DNA is used as starting material for the NGS process.

Calculation of Clonotype (Sequence Cluster) Frequencies from NGS Data

Per sample a large (>10⁵) number of paired reads (nucleotide sequences) is commonly produced by NGS. The read-pairs typically overlap by 40 to 80 bases and are merged read-pair by read-pair to contiguous sequences. These sequences are then assembled into clusters of virtually identical nucleotide sequences with the number of reads per cluster determining the frequency of that cluster. Frequency of a cluster is a measure of the percentage of reads within this sample falling into this cluster.

Clustering works in two rounds: In a first step all reads with 100% nucleotide sequence identity are counted as 1 cluster with the cluster sequence being identical to the read sequence. In the second step clusters are compared among each other and those with
not more than 1 bp mismatch and
where one cluster (cluster A) has at least 20× more reads than the other cluster (cluster B)
   are merged and regarded as identical to cluster A. The nucleotide sequence clusters are regarded as equivalent to clonotypes.

The nucleotide sequence clusters are translated to amino acid sequences (peptides) and tabulated. Each cluster is regarded as one clonotype with a frequency as defined above. The frequency is a direct measure of the frequency of the respective T cell in the sample.

Comparison of TCR Sequence Profiles Between Samples

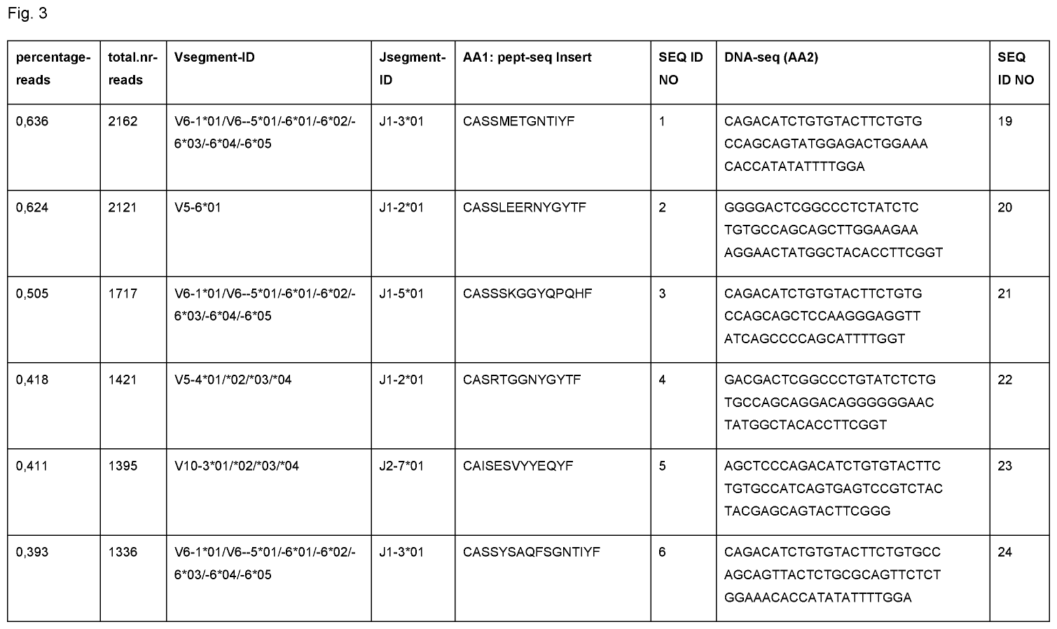

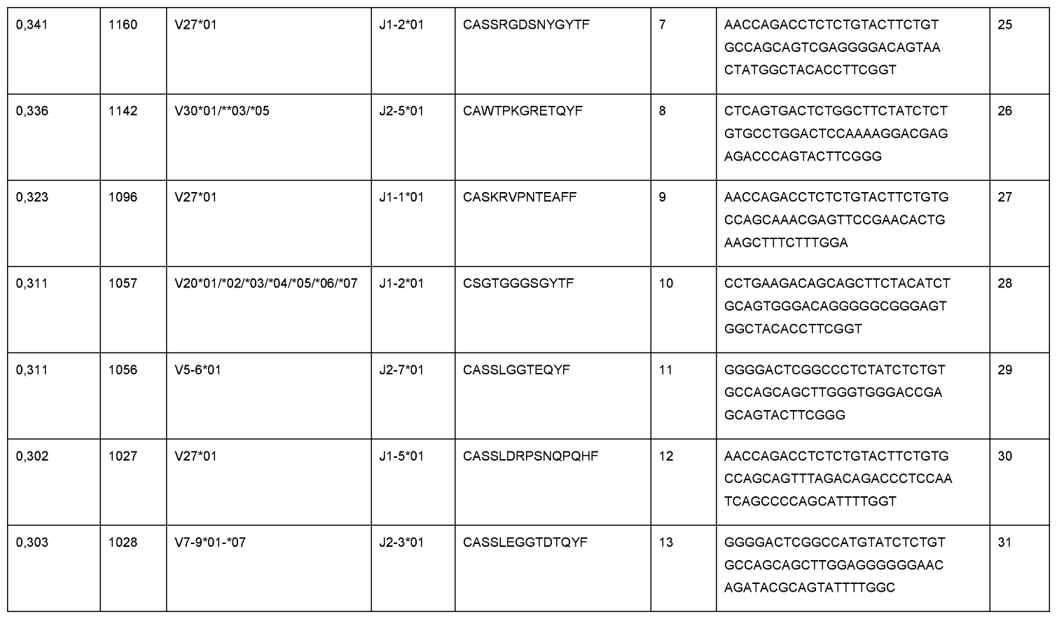

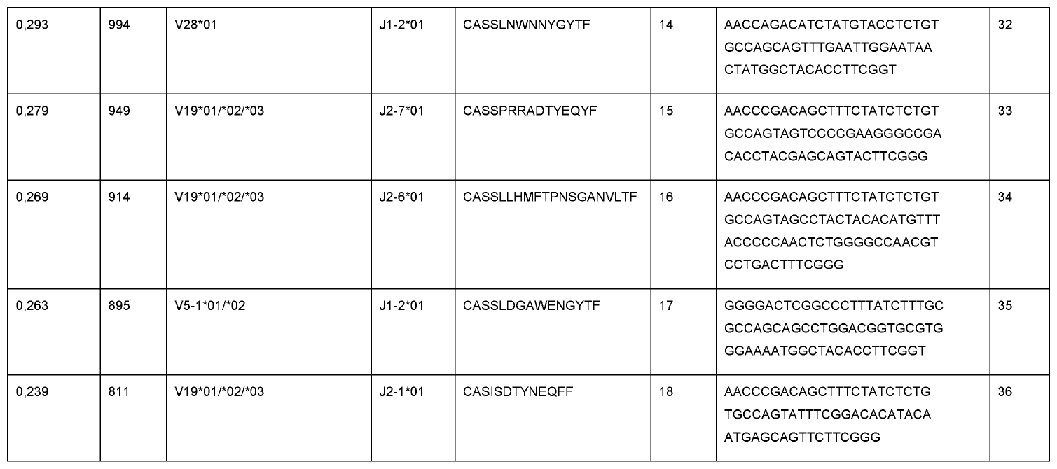

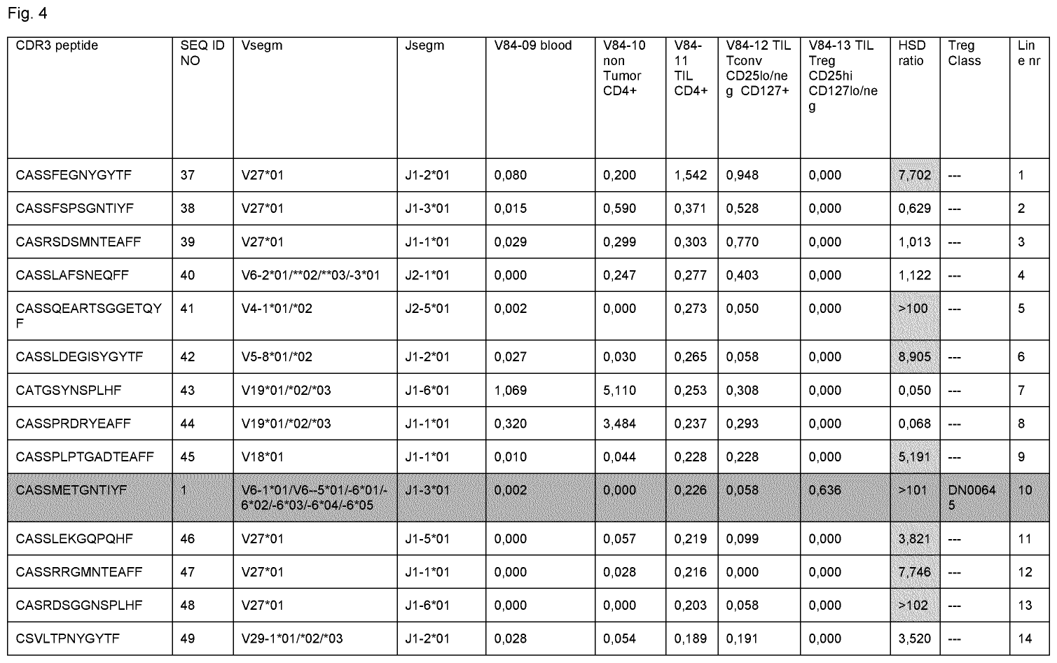

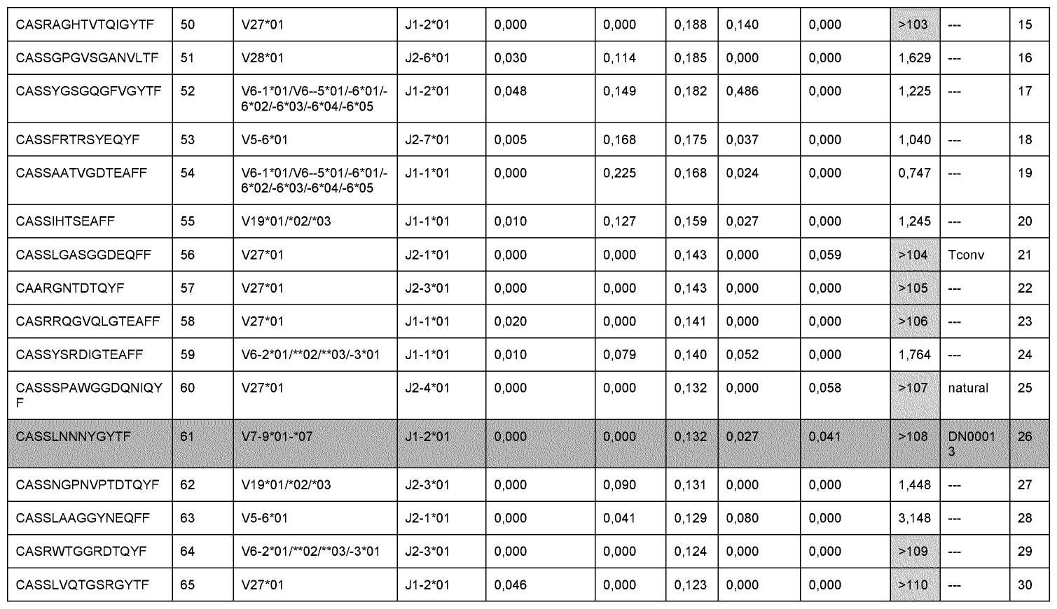

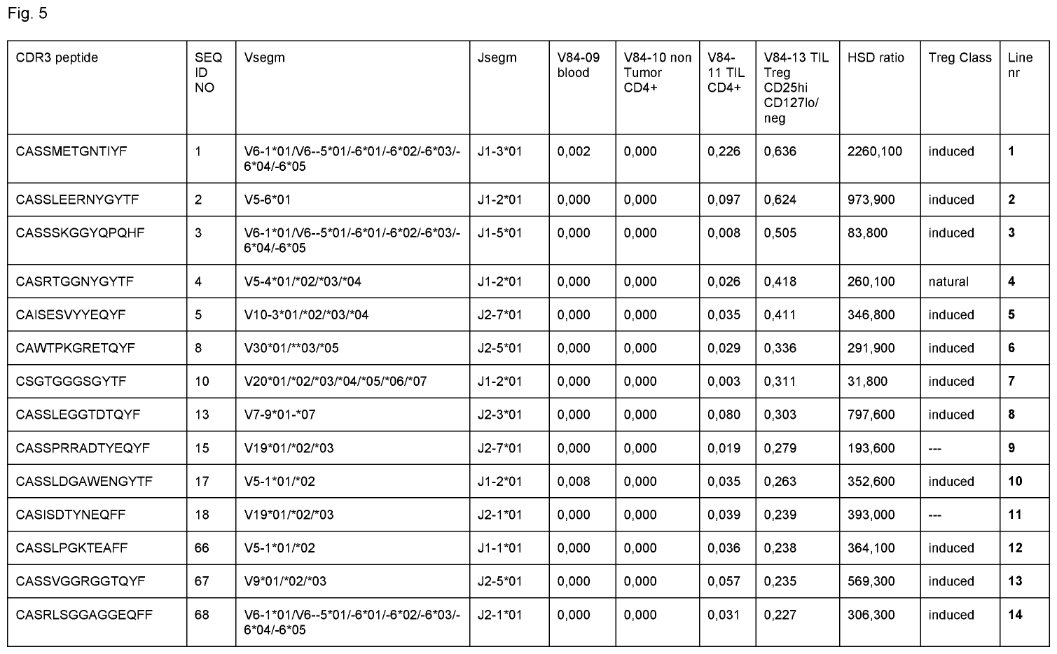

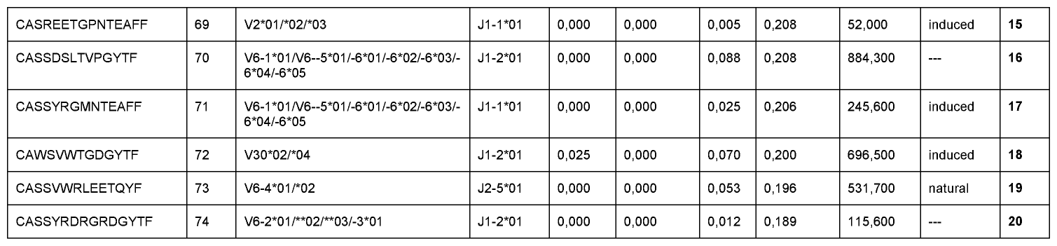

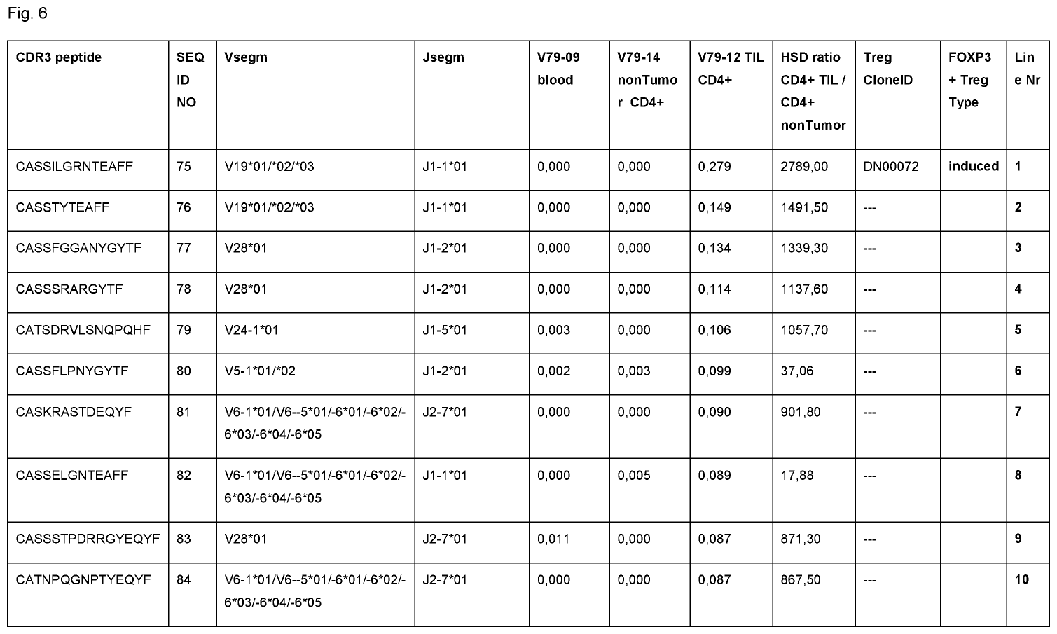

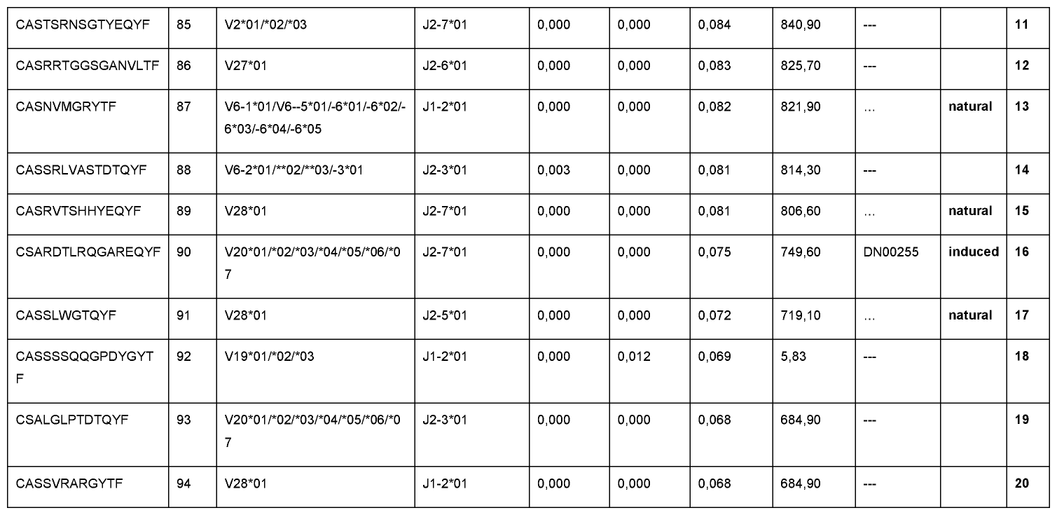

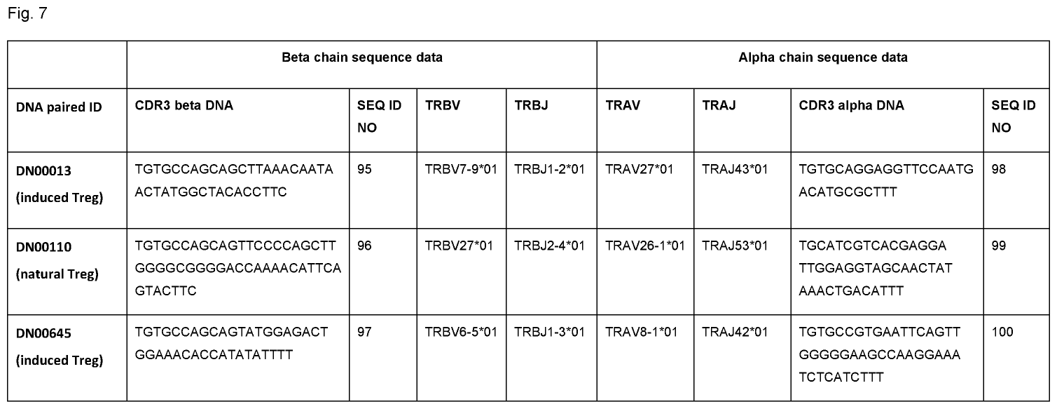

CDR3 amino acid sequences of clonotypes were compared between samples by an identity test procedure, where only sequences without mismatches are accepted as one and the same CDR3 amino acid sequence. The result of a multi-sample comparison is a table with one TCRβ CDR3 amino acid sequence shared by one or more samples per row, each sample is represented by one column containing the respective CDR3 frequencies in that sample (see FIG. 3 representing one sample). Ratios between distinct samples (sharing the same CDR3 amino acid sequence) are calculated by ratio of the respective frequencies.

Identification of Tumour-Specific Treg Clonotypes by Comparative Sequence Analysis

In one exemplary embodiment, tumour-specific Treg clonotypes are identified by the 4-digit score 1100. The score is combined by comparative sequence analysis according to Table 1.

TABLE 1
The scoring table for selection of tumour-specific Treg clonotypes

| | | Tumour | | | | |
|---|---|---|---|---|---|---|
| T cell CDR3 | | specific | | B: Tumour | C: non- | |
| peptide | | Treg | A: Tumour | CD4+ CD25+ | Tumour | D: Blood |
| sequence | Score | clonotype | CD4+ | CD127low/− | CD4+ | CD4+ |
| Seq1 | 1010 | no | 1 | 0 | 1 | 0 |
| Seq2 | 1100 | yes | 1 | 1 | 0 | 0 |
| Seq3 | 1000 | no | 1 | 0 | 0 | 0 |
| . . . | . . . | . . . | . . . | . . . | . . . | . . . |

For each CDR3 peptide sequence (Seq1,2,3, . . . ) simple binary scores are given in each sample (in each column). ‘1’ means, that the respective CDR3 peptide sequence is present in the sample, ‘0’ means it is either absent or found at low levels. The precise definition is given below. The binary scores are combined to a 4-digit score as shown in Table 1. The scoring schema also includes cases, where no blood sample exists, i.e. the column D would be filled with ‘0’, or where no non-tumour sample exist, i.e. the column C would be filled with ‘0’. The binary scores per column (sample type) is defined as follows:

A: score=1: The sequence (Seq1,2,3, . . . ) is among top 100 clonotypes (sorted by their frequency from highest to lowest) and shows an intact open reading frame, i.e. no stop codons or frame shifts are found, otherwise score=0
B: score=1: The sequence (Seq1,2,3, . . . ) is present in the sample. The ratio B/A is greater than 0,5, if B is the frequency in the tumour for TCRs selected as CD4⁺CD25⁺CD127^{low/-} and A is the frequency in the tumour CD4⁺ sample. In all other cases score=0.
C score=0: The sequence (Seq1,2,3, . . . ) is either absent in the non-tumour sample or found identical in the tumour sample, but with a ratio C/A ≤0.2, if C is the frequency in the non-tumour CD4⁺ sample and A is the frequency in the tumour CD4⁺ sample. In all other cases score=1.
D: score=0: The frequency of the sequence (Seq1,2,3, . . . ) is lower than the frequency of the respective sequence in tumour tissue (A), otherwise score=1.

A score of 1100 is needed to identify the respective TCR sequence as tumour specific. Seq2 in Table 1 has a score of 1100 and is identified as a tumour-specific T-reg clonotype.

TCR Alpha/Beta Coupled Sequencing of Tumour-Specific Treg Clonotypes and Synthesis and Cloning of Complete Alpha/Beta Treg TCRs

Paired sequencing of both chains of the T-cell receptor is an essential prerequisite for tailored production of T-cells with target-specific antigen receptors—where the target can be one or several tumour antigens, antigens of viral or microbial origin, antigens related to autoimmune diseases et cetera. As the two chains of any T-cell receptor are encoded on different chromosomes, a sophisticated paired-sequencing approach is required to correlate respective RNAs transcribed form different chromosomes on the single-cell level. There are established technologies to perform paired-sequencing of both chains of the T-cell receptor, with e.g. the technology offered by the company 10×genomics (https://www.10xgenomics.com/solutions/vdj/) being the one which can be implemented in any molecular biological laboratory by using the system's chromium controller for single cell sequencing. Another approach is based on well-plated cell-arrays and deep parallel sequencing followed by statistical analysis (Howie B. et al., High-throughput pairing of T cell receptor α and β sequences. Science Translational Medicine 7, 301 (2015)).

TCRβ CDR3 parts of T-cell receptors identified as tumour specific by the method described above will be paired with their respective α-chains in a separate step by performing single-cell sequencing, usually with a sample of up to 10000 T-cells and selection of the respective paired TCR sequences containing the tumour specific TCRβ CDR3 subsequence. Based on the paired TCR sequence the receptor can be synthesized.

Coupled T Cell Receptor Profiling and FOXP3 Gene Expression Analysis of Treg Cells Using 10×Genomics Technology

Paired TCR single-cell sequencing can be coupled to gene expression analysis to characterize subtypes of T cells and analyze their functional states. FOXP3 is the lineage-defining transcription factor of regulatory T cells. CD4⁺ Treg cells can be differentiated from effector type T cells by high and stable cell surface expression of CD25, absence of CD127 expression and constitutive expression of FOXP3 (CD25^{high}/CD127⁻/FOXP3⁺). To identify Treg among TILs, CD4⁺/CD25⁺/CD127⁻ T cells are sorted by FACS and subjected to combined paired TCR single-cell sequencing and FOXP3 expression analysis using 10×genomic's Chromium™ Single Cell V(D)J Reagent Kit according to the manufacturer's instructions. PCR-amplification of FOXP3 (Genbank Accession AF277993) for sequencing-library generation and subsequent sequencing is achieved by use of in-house designed primers (CTTCTCCTTCTCCAGCACCA (SED ID NO 101); GACACCATTTGCCAGCAGTG (SEQ ID NO 102); TTGAGGGAGAAGACCCCAGT (SEQ ID NO 103)). The 10×technology can detect both natural and induced regulatory T cells. Induced Treg are identified as cells of a given clonotype with identical α/β-TCRs, consisting of FOXP3-expressing and FOXP3-negative T cells.

Alpha- and β-chains of Treg cells are synthesized and cloned into retroviral expression vectors for the stable transduction of fresh effector T cells from autologous tumour patients (see next paragraph).

Gene Transfer of the Isolated and Cloned Tumour-Specific Treg TCRs

This is achieved using a retroviral transduction system (adapted from Voss et al., Adoptive Immunotherapy: Methods and Protocols, Springer Science and Business Media, 2005). Recombinant TCR-transgenic retroviruses are produced using the Phoenix-Ampho packaging cell line. They are transiently transfected with the expression vector encoding the bicistronic construct TCR-beta chain-p2A-TCR-alpha chain, called pMX-puro/βTCR-p2A-αTCR. Co-transfected are the plasmids pHIT60 (from Murine Moloney Leukemia Virus) encoding viral structure proteins and polymerase (reverse transcriptase) as well as pCOLT-GALV (from Gibbon Ape Leukemia Virus) coding for amphotropic envelope proteins. Supernatants containing infectious virus particles can be harvested after 40 to 48 hours. Enriched CD4+ Thelper cells derived from PBMCs of the same patient are infected by incubation with viral supernatant, seeded in 24-well plates and cultured in the incubator (37° C., 5% CO₂) for 24 h. Subsequently, the cells are stimulated with anti-CD3/anti-CD28 in medium supplemented with puromycin in order to expand and enrich TCR-transgenic T-cells. Enrichment is monitored by flow cytometry using TCR-s-chain-specific antibodies. After sufficient expansion, T cells are frozen in aliquots forming a stock of effector T-cells for functional assays.

Detection of Tumor Reactivity of Treg TCR-Transduced Th1-Cells

Treg TCR-transduced Th1-cells (1×10⁵ per well) and tumour cells (1×10⁴ per well), either untreated or pre-treated with 200 IU ml⁻¹ IFN gamma to stimulate expression of MHC class II molecules, are co-cultured in 200 μl RPMI 1640 (Lonza), supplemented with 10% autologous human serum, penicillin and streptomycin (ThermoFisher Scientific). After 48 h, culture supernatants are harvested and analyzed for IFN gamma using the cytometric bead array (BD Biosciences). For flow cytometric detection of intracellular levels of IFN gamma, transduced T cells are co-cultured with tumour cells for 24 h in the presence of Golgi-Plug (BD Biosciences; 1:1,000). Subsequently, cells are stained using surface antibodies to exclude dead cells, and intracellular IFNgamma is detected using the Cytofix/Cytoperm Kit and anti-IFN-γ staining (BD Biosciences), according to the manufacturer's protocol.

Identification of Induced Tumour Infiltrating Treg Sequences as Shown in FIGS. 4 to 7

All data shown in FIGS. 4 and 5 are derived from samples obtained from the same tumor (NSCLC) patient.

The table of FIG. 4 is sorted with respect to descending frequencies in TIL CD4⁺ (col F). Columns A-1 show data obtained by TCR sequencing technology disclosed in WO2014096394A1 (also published as US2015337368 A1, incorporated by reference herein), which uses a set of genomic PCR primers to amplify the beta chain CDR3 region from bulk T-cell DNA.

Each column is from sample preparations of a specific tissue and/or made from cell samples sorted with respect to specific markers.
Blood (col D) is just for reference, without sorting for a dedicated marker
All other cols (E-H) are sorted with respect to CD4⁺ plus evtl. additional markers
Col F vs Col E: Ratio of frequencies per clonotype (ColF/COLE) defines the ratio given in Col 1. A value of >5 is taken as significant for tumor specificity.
ColG vs ColH: By classical sorting with markers CD25 and CD127 with gates set to CD25^{low}/CD127⁺ (T conventional) in ColG and CD25^{high}/CD127^{low/-} in Col H (Treg) one way is established to identify natural Tregs and/or induced Tregs (here significant freqs. in G and H are expected for induced Tregs.
Col I: HSD ratio: TIL CD4⁺/non-Tumor CD4⁺.

10×Genomics' single-cell-sequencing technology allows to identify VDJ-pairing (T-cell receptor, both chains) and gene expression of dedicated genes (here FOXP3) from one cDNA library. The results are single-cell specific, i.e. we can compare sequences cell by cell. In this way distinct cells for one clonotype (same TCRs/VDJ) can be identified which differ by their FOXP3 expression pattern:
a. if one or more FOXP3 positive T-cells, and one or more cells with no FOXP3 expression, bearing essentially the same TCR, are identified, the clonotype is regarded as induced;
b. if one or more FOXP3 positive T-cells, and no other cells within the same VDJ clonotype are identified, it is regarded as natural Treg;
c. If 10× data/FOXP3 expression analysis does not support a. or b., the clonotype may be pure Th-Cell and is not taken into account.

The shaded rows shown in FIG. 4 fulfil criteria a. given above. DN00x stands for (DNA)-group x, which comprises all the Treg- and Th-cells with the same CDR3 regions on beta- and alpha-chains. We can clearly identify two induced Treg clones, which are tumor-specific. In addition, the 10× data deliver the full receptor sequences so that the clone can be produced (gene synthesis of TCRs etc.).

FIG. 5 shows the same data set and patient as for FIG. 4, but sorted with respect to col G and with a filter of the HSD ratio in Col H >5. The 10×analysis was derived from the corresponding T-cell portions with gates [CD25hi CD127 lo/neg]. In rows 4 and 19, one can see natural Tregs (see definition above), almost all other clonotypes of the top20 are induced Tregs—all of them are tumor specific.

FIG. 6 shows Treg analysis based on 10× data alone, and for another patient. The pattern looks different but the ratio plus the 10× analysis delivers 2 induced Tregs and 3 natural Tregs.

FIG. 7 shows the sequences of alpha/beta chains (CDR3 regions only) for the 3 examples of two induced and one natural Tregs in FIG. 4.

1. A method for generating a preparation of tumour-specific T cells, comprising the steps of:
in a tumour T cell isolation step, isolating T regulatory cells from a tumour sample obtained from a patient;
in a tumour T cell sequencing step, obtaining a plurality of nucleic acid sequences encoding tumour infiltrating T cell receptors from said T regulatory cells, yielding a set of tumour TCR sequences;
in a sequence selection step, selecting one or several nucleic acid sequences encoding a tumour-infiltrating T regulatory T cell receptor (tiTreg TCR) from said set of tumour TCR sequences, yielding one or several selected tiTreg TCR sequences;
providing recipient T cells obtained from the patient, wherein said recipient T cells are not T regulatory cells, and
in a gene transfer step, transferring into said recipient T cells a nucleic acid encoding said selected tiTreg TCR under control of a promoter sequence operable in the recipient T cells, thereby yielding a preparation of tumour-specific T cells.

2. The method according to claim 1, wherein in the tumour T cell sequencing step, only sequences encoding T cell receptors originating from CD4+ T cells are sequenced.

3. The method according to claim 1, wherein in the tumour T cell isolation step, T cells are isolated that are characterized by expression of CD4 and
expression of CD25 and low expression of CD127 (CD4⁺CD25⁺CD127^{low}) and/or
FOXP3 (CD4⁺FOXP3⁺).

4. The method according to claim 1, wherein said sequence selection step comprises comparing said tumour TCR sequences
a. among each other, thereby determining the relative incidence (frequency) of any individual sequence, and selecting sequences on account of their incidence; and/or
b. to a set of TCR sequences obtained from T cells (particularly to a set of TCR sequences obtained from CD4⁺ T cells, more particularly CD4⁺CD25⁺ T cells, even more particularly CD4⁺CD25⁺CD127^{low} and/or CD4⁺FOXP3⁺ T cells) obtained from a non-tumour tissue sample of the same patient; and/or
c. to a set of reference TCR sequences,
and wherein sequences are selected that are characterized by high incidence (frequent) in the tumour and/or unique to the set of tumour TCR sequences.

5. The method according to claim 1, wherein prior to the sequence selection step, nucleic acid sequences are translated to amino acid sequences and comparing tumour TCR sequences is performed on the basis of amino acid sequences.

6. The method according to claim 4, wherein said sequence selection step comprises the steps of
aligning the set of tumour TCR sequences in an alignment step;
grouping tumour TCR sequences aligned in the alignment step into a plurality of tumour sample clonotypes, wherein sequences comprised within a particular clonotype exhibit an identical sequence,
particularly wherein comparison of the sequences is performed with respect to the CDR3 sequence tract and a particular clonotype exhibits an identical CDR3 sequence;
determining the number of tumour TCR sequences associated with each clonotype, thereby yielding a clonotype frequency for each of said clonotypes;
selecting a tumour-specific clonotype from said plurality of tumour sample clonotypes, wherein said tumour-specific clonotype is one of the 100 most frequent clonotypes, particularly one of the most 50, 20, 10 or even only 5 of said plurality of tumour sample clonotypes.

7. The method according to claim 6, further comprising
obtaining, from a non-tumour tissue sample obtained from said patient, a plurality of non-tumour tissue T cell receptor sequences; yielding a set of non-tumour tissue TCR sequences;
aligning said plurality of non-tumour tissue T cell receptor sequences;
grouping non-tumour tissue T cell receptor sequences into a plurality of non-tumour tissue clonotypes, wherein T cell receptor sequences comprised within a particular clonotype exhibit a virtually identical or an identical sequence;
determining the number of non-tumour tissue T cell receptor sequences associated with each clonotype, thereby yielding a clonotype frequency for each of said clonotypes;
selecting a tumour-specific clonotype from said plurality of tumour sample clonotypes, wherein the tumour-specific clonotype is absent or exhibits a frequency of <20%, <15%, <10% or <5% within the set of non-tumour tissue TCR sequences.

8. The method according to claim 6, further comprising
obtaining, from a blood sample obtained from said patient, a plurality of blood T cell receptor sequences; yielding a set of blood TCR sequences;
aligning said plurality of blood T cell receptor sequences;
grouping blood T cell receptor sequences into a plurality of blood clonotypes, wherein T cell receptor sequences comprised within a particular clonotype exhibit a virtually identical or an identical sequence;
determining the number of blood T cell receptor sequences associated with each clonotype, thereby yielding a clonotype frequency for each of said clonotypes;
selecting a tumour-specific clonotype from said plurality of tumour sample clonotypes, wherein the tumour-specific clonotype is absent or exhibits a frequency of <20%, <15%, <10% or <5% within the set of blood TCR sequences.

9. The method according to claim 1, wherein a nucleic acid sequence encoding a tumour-infiltrating T regulatory T cell receptor (tiTreg TCR) is selected from said set of tumour TCR sequences if
a. the clonotype is present in both the tiTreg (CD4⁺CD25^{high}CD127^{low} or CD4⁺FOXP3⁺) and tiTconv (CD4⁺CD25^{low}CD127⁺ or CD4⁺FOXP3⁻) populations, or
b. single cell expression analysis detects FOXP3 expressing and FOXP3 negative cells among tumour-infiltrating CD4⁺ cells.

10. The method according to claim 1, wherein obtaining T cell receptor sequences comprises the steps of
a. isolating T cells from said tumour sample, non-tumour tissue sample and blood sample and isolating nucleic acid from the isolated T cells, and
b. conducting a nucleic acid amplification reaction that specifically amplifies T cell receptor nucleic acid sequences.

11. The method according to claim 9, wherein the nucleic acid amplification reaction specifically amplifies a sequence encoding the CDR3 region of a chain of the T cell receptor, particularly the CDR3 region of the alpha chain or the beta chain of the T cell receptor.

12. The method according to claim 1, wherein recipient T cells are selected from the group comprising cytotoxic T cells and T helper cells, particularly wherein the method comprises a step of
a. selecting T helper cells from a sample obtained from the patient, yielding an enriched T helper cell preparation, or
b. selecting cytotoxic T cells from a sample obtained from the patient, yielding an enriched cytotoxic T cell preparation
and subjecting the enriched T cell preparation to the gene transfer step.

13. The method according to claim 1, wherein said recipient T cells have been prepared by depletion of CD8⁺ T cells and CD4⁺ regulatory T cells, particularly from a lymphocyte preparation of said patient.

14. The method according to claim 1, wherein the gene transfer step is preceded by a transgene generating step, wherein the nucleic acid encoding said selected tiTreg TCR is under control of a promoter sequence is generated by
a. separating a plurality of T regulatory cells obtained from said tumour sample into single cells;
b. obtaining a plurality of complete TCR sequence sets, each TCR sequence set comprising a full TCR alpha and TCR beta polypeptide sequence from said plurality of cells;
c. assigning the selected tiTreg TCR sequence to a complete TCR sequence set, yielding a complete tiTreg TCR sequence set;
d. inserting sequences encoding the full TCR alpha and TCR beta polypeptide sequence of said complete tiTreg TCR sequence set determined in the preceding step into a gene expression construct.

15. A preparation of transgenic T cells, obtained by a method according to claim 1.

16. A preparation of transgenic T cells, obtained by a method according to claim 1, for use in treatment, or prevention of recurrence, of cancer.
